(19) United States
(12) Patent Application Publication
(10) Pub. No.: US 2022/0233473 A1
(43) Pub. Date: Jul. 28, 2022

(54) INJECTABLE SOLUTION OF NOREPINEPHRINE

(71) Applicant: Cumberland Pharmaceuticals Inc., Nashville, TN (US)

(72) Inventors: Leo PAVLIV, Cary, NC (US); Andrew VILA, Nashville, TN (US)

(73) Assignee: Cumberland Pharmaceuticals Inc., Nashville, TN (US)

(21) Appl. No.: 17/584,714

(22) Filed: Jan. 26, 2022

Related U.S. Application Data
(60) Provisional application No. 63/142,722, filed on Jan. 28, 2021.

(51) Int. Cl. A61K 31/137; A61K 9/00

(52) U.S. Cl. CPC A61K 31/137; A61K 9/0019

(57) ABSTRACT

A ready-to-administer stable injectable norepinephrine solution comprising water, norepinephrine, and a tonicity agent. The solution has a pH from 3.7 to 4.3 and is substantially free of chelating agents and antioxidants.

### FIELD OF THE INVENTION

The present invention relates to stable, ready-to-administer injectable solutions comprising norepinephrine.

### BACKGROUND OF THE INVENTION

Norepinephrine (NE) is a sympathomirmetic amine which functions as a peripheral vasoconstrictor (alpha-adrenergic action) and as an inotropic stimulator of the heart and dilator of coronary arteries (beta-adrenergic action). It is also known as l-arterenol, levarterenol or l-norepinephrine or noradrenaline. A salt of norepinephrine, norepinephrine bitartrate, is chemically (−)-α-(aminomethyl)-3,4-dihydroxybenzyl alcohol tartrate (1:1) (salt) monohydrate and has the following structural formula:

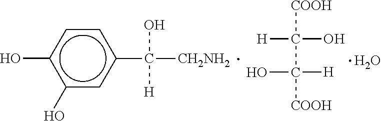

Injectable forms of norepinephrine bitartrate are approved by the U.S. Food and Drug Administration for blood pressure control in certain acute hypotensive states (e.g., pheochromocytomectomy, sympathectomy, poliomyelitis, spinal anesthesia, myocardial infarction, septicemia, blood transfusion, and drug reactions) and as an adjunct in the treatment of cardiac arrest and profound hypotension.

These commercially available injectable norepinephrine products are supplied in a concentrated form and must therefore be diluted prior to administration. Following dilution, these norepinephrine formulations cannot be stored for long periods and are normally discarded within one day after reconstitution. Consequently, these concentrated formulations are at risk for both microbial contamination as well as dilution errors. There is therefore a significant need for a ready-to-administer injectable norepinephrine solution to improve patient safety and ease of use, especially in instances where the product is needed for administration urgently, for instance to treat cardiac arrest.

At the same time, norepinephrine is known to be unstable and susceptible to oxidation and degradation in the presence of oxygen. This is particularly true when norepinephrine is present in dilute aqueous solutions. The degradation is undesirable, as it results, for example, in loss of titer of the active ingredient, formation of compounds which have undesirable physiological effects, and the appearance of a dark color, which makes the solution offensive and unmarketable.

Therefore, a significant need exists for injectable norepinephrine formulations that are both stable and ready-to-administer, as well as methods of manufacturing and using such norepinephrine formulations.

### SUMMARY OF THE INVENTION

The invention is directed to a ready-to-administer stable injectable norepinephrine solution comprising water; norepinephrine, or a pharmaceutically acceptable salt thereof, in a concentration of from 0.04 to 0.16 mg/ml; and a tonicity agent, wherein the pH of the solution is from 3.7 to 4.3, and wherein the solution is substantially free of chelating agents and antioxidants. In another embodiment, the ready-to-administer stable injectable norepinephrine solution consists of water; norepinephrine, or a pharmaceutically acceptable salt thereof, in a concentration of from 0.04 to 0.16 mg/ml; and a tonicity agent, wherein the pH of the solution is from 3.7 to 4.3, and wherein the solution is substantially free of chelating agents and antioxidants. The ready-to-administer stable injectable norepinephrine solution is contained within a first container. The first container may be optionally further packaged in a second container.

Accordingly, one embodiment is a process for producing a ready-to-administer stable injectable norepinephrine solution comprising:
(a) combining a tonicity agent, water, and norepinephrine, or a pharmaceutically acceptable salt thereof, to obtain a composition with a concentration of norepinephrine of from 0.04 to 0.16 mg/mL;
(b) filling the composition into a first container; and
(c) sterilizing the composition to sterility to obtain the ready-to-administer stable injectable norepinephrine solution, wherein the pH of the solution is from 3.7 to 4.3 and wherein the solution is substantially free of chelating agents and antioxidants.
   The first container may be optionally further packaged in a second container.

Another embodiment is methods of controlling Hood pressure and treating cardiac arrest, profound hypotension, and other conditions where a peripheral vasoconstrictor, inotropic stimulator of the heart, or dilator of coronary arteries is desired comprising administering to a patient in need thereof a ready-to-administer stable injectable norepinephrine solution comprising water; norepinephrine, or a pharmaceutically acceptable salt thereof, in a concentration of from 0.04 to 0.16 mg/ml; and a tonicity agent, wherein the pH of the solution is from 3.7 to 4.3, and wherein the solution is substantially free of chelating agents and antioxidants. The ready-to-administer stable injectable norepinephrine solution may be administered directly from a first container, without any reconstitution or dilution.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that the foregoing general description and the following detailed description are exemplary and explanatory only and are not restrictive of the invention, as claimed.

### DETAILED DESCRIPTION OF THE INVENTION

The term “norepinephrine,” as used herein covers norepinephrine as well as its pharmaceutically acceptable salts thereof, including organic salts, such as norepinephrine bitartrate, and mineral salts, such as norepinephrine hydrochloride.

The term “ready-to-administer” as used herein means that the norepinephrine solution is sterile and suitable for direct intravenous infusion or injection and no intermediate steps of dilution or reconstitution are required before administration of the solution to the patient. The norepinephrine solution can be directly administered from the container of the dosage form. The term “ready-to-administer” is synonymous with “ready-to-infuse” or “ready-to-inject” and can be distinguished from the term “ready-to-use” solution. The term “ready-to-use” in the art includes preconcentrates which require dilution with an aqueous diluent fluid such as water for injection or saline before administration. The term “ready-to-administer” is also distinguished from lyophilized products that require two steps, a first step of reconstitution to form a preconcentrate and then a second step where the preconcentrate is subjected to dilution with an aqueous infusion fluid. The ready-to-administer dosage form avoids the inconvenience of reconstituting or diluting a concentrated formulation into infusion diluents prior to infusion, as well as eliminates the risk of any potential calculation or dilution error as well as risk of microbiological contamination during handling.

As used herein, the term “stable” means that the norepinephrine solution is physically as well as chemically stable as demonstrated by compliance to acceptable specification upon storage for a prolonged period of time, such as for at least 3 months, 6 months, 12 months, 18 months, or at least 24 months when stored at room temperature (about 25° C.) and/or for a period of at least 6 months when stored at accelerated stability conditions (about 40° C.). For instance, the solution of norepinephrine remains physically stable, with no precipitation or crystallization or color change upon storage when stored at these conditions. Suitably, the solution of norepinephrine remains chemically stable when stored at these conditions when various parameters such as the norepinephrine content and content of related substances, i.e., known and unknown impurities, remains within specified limits such as those specified according to ICH guidelines, upon storage. Suitably, the concentration of L-norepinephrine remains within 90-110% by weight when stored at these conditions. The concentration of L-norepinephrine may be measured using a chiral IPLC method (Stepensky et al. (2004) Journal of Pharmaceutical Sciences, Vol. 93, No. 4, 969-980). Suitably, the total impurities remain below 2.0% or below 1.0% when stored at these conditions. The amount of chemical impurities may be measured using the United States Pharmacopeia (USP) Assay method for Norepinephrine Bitartrate (United States Pharmacopeia, The National Formulary, “Norepinephrine Bitartrate Injection,” (2018)).

The ready-to-administer norepinephrine solution comprises water, norepinephrine, or a pharmaceutically acceptable salt thereof, and a tonicity agent. In one embodiment, the pharmaceutically acceptable salt of norepinephrine is norepinephrine bitartrate. Norepinephrine or its pharmaceutically acceptable salt will typically be present in the solution at a concentration of less than 0.20 mg/mL or less than 0.16 mg/mL. For instance, the norepinephrine may be present at a concentration of from 0.01 mg/mL to 0.20 mg/mL, from 0.04 mg/mL to 0.20 mg/mL, or from 0.04 mg/mL to 0.16 mg/mL. The norepinephrine may be present at a concentration of 0.04 mg/mL, 0.08 mg/mL or 0.16 mg/mL.

Norepinephrine is known to exist in two different stereoisomeric forms: L (−) isomer and D (+) isomer. The L isomer is reported to have better affinity at various receptors and thus, is more potent than the D isomer. The D isomer has minimal to almost negligible activity. As will be readily appreciated, the norepinephrine for preparation of the aqueous solutions of the invention is L-Norepinephrine, or enantiomerically pure (i.e., at least 98% L-isomer) norepinephrine. In other aspects, isomeric purity can also be from 95% to 98% L-isomer norepinephrine.

The water used in the norepinephrine solutions may be degassed or deaerated, distilled, sterile, pyrogen-free, or water-for-injection (WFI). According to some embodiments, the deoxygenated or degassed water is obtained by blowing or bubbling an inert gas current, such as nitrogen, argon, or mixtures thereof.

The tonicity agent used in the norepinephrine solutions may be selected from, but is not limited to, sodium chloride, potassium chloride, calcium chloride, mannitol, glycerol, sorbitol, propylene glycol, dextrose, sucrose, and mixtures thereof. According to one embodiment, the norepinephrine solution comprises sodium chloride as a tonicity agent. An osmometer can be used to check and adjust the amount of tonicity agent to be added to obtain the desired osmolality of the norepinephrine solution. The norepinephrine solution may have an osmolality in the range of from 250 to 375 mOsm/kg, from 260 to 340 mOsm/kg, or from 270 to 330 mOsm/kg. For instance, sodium chloride may be used in an amount ranging from 0.3% w/v to 1.0% w/v or from 0.5% w/v to 1.0%. In one embodiment, sodium chloride is present in the norepinephrine solution in an amount of 0.9% w/v.

The norepinephrine solution is substantially free of chelating agents and antioxidants. The term “substantially free” as used herein means that chelating agents and antioxidants, if present, are present as impurities. In other words, chelating agents and antioxidants are not added to the solution, but may be present otherwise, for instance as an impurity or undesired contaminant, typically in an amount less than 0.005% by weight, as determined by HPLC-MS. Examples of chelating agents include, but are not limited to, an aminopolycarboxylic acid such as ethylenediaminetetraacetic acid, commonly referred to as EDTA, diethylenetriaminepentaacetic acid (DTPA), triethylenetetraaminehexaacetic acid (TTHA), trans-1,2-diaminocyclohexane-N,N,N′,N′-tetraacetic acid (CDTA), ethylenediaminedisuccinic acid (EDDS), a bicarboxylic acid, and a tricarboxylic acid. Examples of antioxidants include, but are not limited to, sodium sulfite, sodium bisulfite, sodium metabisulfite, butylated hydroxyl anisol, ascorbic acid, propyl gallate, vitamin E, alpha-tocopherol, and butylated hydroxyl toluene.

Accordingly, the norepinephrine solution is substantially free of EDTA, DTPA, sodium metabisulfite, butylated hydroxyl anisol, and combinations thereof.

Suitably, the norepinephrine solution has a pH in the range of from 3.7 to 4.3. In one embodiment, the pH of the norepinephrine solution is from 3.7 to 4.0. In yet another embodiment, the pH of the norepinephrine solution is from 4.0 to 4.3. In still other embodiments, the norepinephrine solution may have a pH of 3.5, 3.6, 3.7, 3.8, 3.9, 4.0, 4.1, 4.2, 4.3, 4.4 or 4.5. The pH of the norepinephrine solution may be adjusted in the desired range by use of a pH adjusting agent. The pH adjusting agent that may be used includes, but is not limited to, sodium hydroxide, hydrochloric acid, sulfuric acid, citric acid, acetic acid, tromethamine, potassium hydroxide, and mixtures thereof. It may not be necessary to add a pH adjusting agent to obtain the desired pH of the norepinephrine solution. Accordingly, in one embodiment, the norepinephrine solution is substantially free of pH adjusting agents.

The ready-to-administer norepinephrine solution may contain norepinephrine as the only active ingredient. The norepinephrine solution may be substantially free of active ingredients other than norepinephrine that have the same or similar activity as norepinephrine. For instance, the norepinephrine solution may be substantially free of peptides, such as neuropeptide Y (NPY) or peptide YY (PYY), and/or compounds other than norepinephrine that are useful in controlling blood pressure or in the treatment of cardiac arrest or profound hypotension.

The ready-to-administer norepinephrine solution may be contained within a first container. The first container may be optionally further packaged in a second container. In one embodiment, either or both of the first container and the second container is designed to protect the solution of norepinephrine from light, oxygen, or both.

The second container may comprise a suitable pouch, such as an aluminum pouch or a carton packaging. The second container may completely surround the first container. In one embodiment, the second container is designed to protect the norepinephrine solution from light, oxygen, or both. For instance, the second container may be composed of a suitable light protective material such as aluminum. In another embodiment, the space between the first container and the second container is occupied with an inert gas. The inert gas may be used to flush out or replace the air from the space between the first container and the light protective second container. The inert gas that may be used includes, but is not limited to, nitrogen, argon and helium. In one specific embodiment, the second container comprises an aluminum pouch containing an oxygen scavenger. In another embodiment, the space between the first container and second container is occupied with an inert gas, such as nitrogen.

The first container may be designed such that the solution of norepinephrine is ready-to-administer to the patient. In other words, the solution of norepinephrine may be directly administered to the patient, without any reconstitution or dilution. The first container may be composed of one or more layers of a suitable material such as plastic or polymeric material. Suitably, such materials may include but are not limited to, polyolefin polymers, polyethylene, polypropylene; cyclo olefin polymers, cyclo olefin copolymers, polypropylene based polyolefin polymers; polycarbonates; modified polyolefin-polyethylene polymers or styrene-polyolefin based polymers and block co-polymers thereof. These plastic materials of the container may further have one or more outer layers which may be made up of polyamide, modified polyolefin, polypropylene, styrene-polyolefin based polymers and block co-polymers thereof and the like. In one specific embodiment, the first container is composed of polypropylene.

In one embodiment, the first container does not have material that contains borate or boron. According to another embodiment, the first container may be composed of non-glass components. Suitably, the material of the first container may be transparent which makes it possible to carry out visual inspection of the drug solution prior to and during administration of the norepinephrine solution.

In one embodiment, the first container is a pre-filled syringe. The syringe may be further packed in a second container to protect from light. The second container may comprise a pouch, such as an aluminum pouch, and/or a carton packaging. The pouch may further contain an oxygen scavenger. In this embodiment, the volume of the norepinephrine solution in the pre-filled syringe may vary from about 50 ml to about 100 ml.

In another embodiment, the first container is an infusion (IV) bag. The infusion bag may be further packed in a second container to protect from light. The second container may comprise a pouch, such as an aluminum pouch, and/or a carton packaging. The pouch may further contain an oxygen scavenger. The volume of the norepinephrine solution in the first container, such as an infusion bag, may vary from about 50 mL to about 500 mL. In one embodiment, the volume in each bag is from 100 mL to 250 mL. The volume of the norepinephrine solution in each bag may be, for instance, 50 mL, 100 mL, 150 mL, 200 mL, 250 mL, 300 mL, 350 mL, 400 mL, 450 mL or 500 mL.

The first container has a volume and dimensions such that it allows use of the norepinephrine solution such that the solution can be directly infused to the patients without any step of reconstitution or dilution. The concentration and volume may be such that for a patient with an average body surface area, only one unit of the first container is sufficient to deliver the prescribed dose of norepinephrine.

The ready-to-administer stable norepinephrine solutions may be produced by combining a tonicity agent, water, and norepinephrine, or a pharmaceutically acceptable salt thereof. The tonicity agent, water, and norepinephrine, or a pharmaceutically acceptable salt thereof, may be combined in any order. For instance, the norepinephrine, or a pharmaceutically acceptable salt thereof, may be dissolved in an aqueous solution of the tonicity agent. In one embodiment, norepinephrine bitartrate is dissolved in 0.9% normal saline. Alternatively, the norepinephrine, or a pharmaceutically acceptable salt thereof, may be dissolved in water, followed by adding a tonicity agent to the norepinephrine solution. Optionally, the pH of the norepinephrine solution may be measured and, if necessary, adjusted by the addition of a pH-adjusting agent.

The norepinephrine solution may be sparged with an inert gas to reduce oxygen levels. In one embodiment, the norepinephrine solution is sparged until the dissolved oxygen level is less than 4.0 ppm, less than 2.0 ppm, or less than 1.0 ppm. The insert gas may be nitrogen, argon, or helium. For example, the norepinephrine solution may be sparged with nitrogen gas until the dissolved oxygen level is less than 1.0 ppm.

The norepinephrine solution may be filtered, for instance through a membrane filter, before being filled into a first container. The speed of the norepinephrine solution through the filter may be increased by blowing a current of inert gas that acts as a carrier. Suitable filters are those used in the pharmaceutical technology for the preparation of sterile injectable solutions. Optionally, an inert gas, such as nitrogen, may be added to the first container prior to sealing the first container.

The first container may be optionally further packaged in a second container. In one embodiment, the norepinephrine solution is filled into an infusion (IV) bag and the infusion bag is then sealed into an aluminum pouch. The air in the space between the first and second containers, may be displaced prior to sealing of the second container, for instance, by a stream of inert gas.

The norepinephrine solution may be sterilized at any point or at multiple points during the manufacturing process. For instance, the norepinephrine solution may be sterilized after it is filled into the first container. Alternatively, the norepinephrine solution may be sterilized after the first container is packaged into a second container. In yet a further embodiment, the norepinephrine solution may be sterilized before it is filled into the first container. Combinations of any of the foregoing are also possible. Sterilization may be achieved by suitable techniques such as filtration sterilization, heat sterilization, and radiation sterilization. In one embodiment, the norepinephrine solution is sterilized by autoclaving after the solution is filled into a first container. In another embodiment, the norepinephrine solution is sterilized by autoclaving after the first container is sealed in the second container.

The inventors have found that the ready-to-administer norepinephrine solutions are stable, without having to undergo freeze-drying, reconstitution, dilution, or the like, with a concentration of active L-isomer of norepinephrine greater than or equal to 98%. Moreover, the concentration of active L-isomer of norepinephrine is maintained at greater than or equal to 90% when stored at room temperature conditions for up to 3 months, 6 months, 12 months, 18 months, or 24 months.

Norepinephrine administration has been shown to be useful for blood pressure control in certain acute hypotensive states (e.g., pheochromocytomectomy, sympathectomy, poliomyelitis, spinal anesthesia, myocardial infarction, septicemia, blood transfusion, and drug reactions) and as an adjunct in the treatment of cardiac arrest and profound hypotension. Accordingly, the present invention is also directed to methods of controlling blood pressure, treating cardiac arrest, and treating profound hypotension comprising using the ready-to-administer stable injectable norepinephrine solutions.

The ready-to-administer stable injectable norepinephrine solution may be administered by intravenous injection. The norepinephrine solution is ready-to-administer, such that it may be directly infused to a patient from the first container without any step of reconstitution or dilution.

Individual variation occurs in the dose required to attain and maintain an adequate blood pressure. Accordingly, dosage of the norepinephrine solution should be titrated according to the response of the patient. After observing the response to an initial dose of from 8 mcg to 12 mcg of norepinephrine per minute, the rate of flow may be adjusted to establish and maintain a low normal blood pressure (usually 80 mm Hg to 100 mm Hg systolic) sufficient to maintain the circulation to vital organs. In previously hypertensive patients, the blood pressure should be raised no higher than 40 mm Hg below the preexisting systolic pressure. The average maintenance dose ranges from 2 mcg to 4 mcg of norepinephrine per minute. Much larger daily doses (as high as 68 mg norepinephrine) may be necessary if the patient remains hypotensive. The infusion of the norepinephrine solution should be continued until adequate blood pressure and tissue perfusion are maintained without therapy.

### EXAMPLES

The following examples are not meant to be limiting and represent certain embodiments of the present invention.

### Example 1

Table 1 shows stability data for the current commercial product, LEVOPHED® (norepinephrine bitartrate), which is available as a concentrated 4 mg/4 mL solution packaged in a vial with a nitrogen headspace. It was found that the stability declines steadily over 12 months. This was observed by two separate analytical methods, which show (1) an increase of chemical impurities and also (2) an increase of the inactive D-isomer (only the L-isomer is active), as measured by a chiral HPLC method. The method for testing chemical impurities was the USP Assay method for Norepinephrine Bitartrate (United States Pharmacopeia, The National Formulary, “Norepinephrine Bitartrate Injection,” (2018)) and the method for detecting L-isomer was a chiral HPLC method (Stepensky et al. (2004) Journal of Pharmaceutical Sciences, Vol. 93, No. 4, 969-980). The results show an over 9% loss of activity after 12 months at 25° C. There is a much more significant loss of activity at 40° C., resulting in only 86% of the active form after storage for 3 months at 40° C.

TABLE 1
Stability data for LEVOPHED ® (norepinephrine bitartrate)
at 25° C. and 40° C.

| | | | | | Storage at |
|---|---|---|---|---|---|
| | | Storage at 25° C. | | | 40° C. |
| | Initial* | 1 Month | 3 Month | 12 Month | 3 Month |
| Purity (%) | | | | | |
| Norepinephrine | 99.00 | 98.75 | 97.61 | 95.14 | 94.16 |
| Total Impurities/RS | 1.00 | 1.25 | 2.39 | 4.86 | 5.84 |
| Chiral purity of | | | | | |
| Norepinephrine (%) | | | | | |
| L-Norepinephrine | 95.27 | 97.43 | 96.04 | 91.18 | 85.91 |
| D-Norepinephrine | 1.81 | 1.23 | 1.80 | 4.38 | 8.30 |

*Initial stability is estimated to be 5 months from date of manufacture

It is generally accepted that injectable pharmaceuticals are most stable in glass vials or ampules where they are hermetically sealed and the glass has less ability to interact with the product than packaging in ready-to-use bags made of various plastics. This data demonstrates, however, that even in glass, norepinephrine is highly subject to degradation.

### Example 2

Solutions of norepinephrine were made at concentrations of 0.04, 0.08, and 0.16 mg/mL in 0.9% normal saline. Norepinephrine bitartrate (>99.9%) from GYMA Laboratories of America, Inc., was used to make the norepinephrine solutions. The norepinephrine bitartrate powder was added to a premade solution of sodium chloride (0.9% w/v) obtained from Baxter IV bags. The final pH of the solutions were approximately 4.3, 4.0, and 3.8 for the 0.04, 0.08, and 0.16 mg/mL norepinephrine solutions, respectively. Oxygen levels were reduced by sparging the norepinephrine solutions with nitrogen until the dissolved oxygen levels were <1.2 ppm. The nitrogen-sparged solutions were then filtered through a 0.2 μm PVDF membrane, sparged with nitrogen again to lower the dissolved oxygen levels to <1.0 ppm, and then filled into polypropylene IV bags (Technoflex) or glass vials.

Some of the IV bags (“Nitrogen-sparged”) were then sealed into 5.5×12″ aluminum pouches (Amcor Flexibles, Catalog number RFP-073). The air in the pouches was displaced by a stream of nitrogen from a compressed nitrogen cylinder prior to heat sealing. The solution in the remaining IV bags (“Ambient air”) was allowed to equilibrate to dissolved oxygen levels between ˜5 and 10 ppm and were not sealed into a foil pouch. Some of the IV bags were terminally sterilized by autoclaving, while others were not. The terminal sterilization process comprised heating the samples at 121° C. for 15 minutes after the IV bags were filled for the Ambient air samples or after the IV bags were filled and then sealed in foil pouches for the Nitrogen-sparged containing samples. The Ambient air IV bags were wrapped loosely in aluminum foil to prevent them from bursting during the autoclave cycle.

The norepinephrine solutions filled into the vials were either sealed with a nitrogen headspace (“Nitrogen-sparged”) or allowed to equilibrate to dissolved oxygen levels of ˜5 ppm before filling and a nitrogen headspace was not added (“Ambient air”). Some of the vials were terminally sterilized by autoclaving, as described above, while others were not.

All of the IV bags and vials were placed on stability and tested for (1) chemical impurities using the USP Assay method for Norepinephrine Bitartrate and (2) L-isomer content using the chiral HPLC method, as described above. As shown in the Tables below, the norepinephrine solutions in IV bags were generally more stable than the solutions filled into glass vials. It was found that nitrogen-sparged norepinephrine solutions were more stable in an autoclaved IV bag compared to an autoclaved vial. Samples containing autoclaved norepinephrine solutions but with no nitrogen sparging showed the same trend. Non-autoclaved samples with no nitrogen sparging showed the opposite trend where norepinephrine solutions in vials were more stable than IV bags, as expected. Overall, the data shows that nitrogen-sparged norepinephrine solutions were more stable in IV bags compared to both commercially available product and also the norepinephrine solutions prepared and filled into vials.

TABLE 2
Stability data at 25° C. for 0.16 mg/mL Norepinephrine in vials and IV bags that were
nitrogen sparged and autoclaved.

| | Storage at 25° C. | | | | | | |
|---|---|---|---|---|---|---|---|
| | Vials | | | IV bags | | | |
| | Initial | 3 Month | 6 Month | Initial | 3 Month | 6 Month | 24 Month |
| Purity (%) | | | | | | | |
| Norepinephrine | 99.75 | 97.44 | 95.66 | 100.00 | 99.79 | 99.90 | 98.87 |
| Total Impurities/RS | 0.25 | 2.57 | 4.34 | 0.0 | 0.2 | 0.1 | 1.14 |
| Chiral purity of | | | | | | | |
| Norepinephrine | | | | | | | |
| (%) | | | | | | | |
| L-Norepinephrine | 97.91 | 95.50 | 94.80 | 99.62 | 98.87 | 98.64 | 94.66 |
| D-Norepinephrine | 1.83 | 3.29 | 3.97 | 0.21 | 0.88 | 1.10 | 4.48 |

TABLE 3
Stability data at 40° C. for 0.16 mg/mL Norepinephrine in vials
and IV bags that were nitrogen sparged and autoclaved.

| | Storage at 40° C. | | | |
|---|---|---|---|---|
| | Vials | | IV bags | |
| | Initial | 6 Month | Initial | 6 Month |
| Purity (%) | | | | |
| Norepinephrine | 99.75 | 85.92 | 100.00 | 97.64 |
| Total Impurities/RS | 0.25 | 14.07 | 0.0 | 2.37 |
| Chiral purity of | | | | |
| Norepinephrine (%) | | | | |
| L-Norepinephrine | 97.91 | 81.64 | 99.62 | 91.54 |
| D-Norepinephrine | 1.83 | 13.47 | 0.21 | 7.87 |

TABLE 4
Stability data at 25° C. for 0.16 mg/mL Norepinephrine in vials and IV bags that were
nitrogen sparged and not autoclaved.

| | Storage at 25° C. | | | | | | | |
|---|---|---|---|---|---|---|---|---|
| | Vials | | | IV bags | | | | |
| | | 3 | 6 | | 3 | 6 | 12 | 18 |
| | Initial | Month | Month | Initial | Month | Month | Month | Month |
| Purity (%) | | | | | | | | |
| Norepinephrine | 99.91 | 99.50 | 99.45 | 100.00 | 99.82 | 99.70 | 99.59 | 99.26 |
| Total | 0.09 | 0.49 | 0.54 | 0.0 | 0.18 | 0.3 | 0.41 | 0.74 |
| Impurities/RS | | | | | | | | |
| Chiral purity of | | | | | | | | |
| Norepinephrine | | | | | | | | |
| (%) | | | | | | | | |
| L- | 99.64 | 98.91 | 98.43 | 99.90 | 99.46 | 99.16 | 98.55 | 96.86 |
| Norepinephrine | | | | | | | | |
| D- | 0.08 | 0.54 | 0.98 | n.d. | 0.41 | 0.71 | 1.26 | 2.65 |
| Norepinephrine | | | | | | | | |

n.d. = not detected

TABLE 5
Stability data at 40° C. for 0.16 mg/mL Norepinephrine in vials
and IV bags that were nitrogen sparged and not autoclaved.

| | Storage at 40° C. | | | |
|---|---|---|---|---|
| | Vials | | IV bags | |
| | Initial | 6 Month | Initial | 6 Month |
| Purity (%) | | | | |
| Norepinephrine | 99.91 | 86.18 | 100.00 | 97.83 |
| Total Impurities/RS | 0.09 | 13.81 | 0.0 | 2.19 |
| Chiral purity of | | | | |
| Norepinephrine (%) | | | | |
| L-Norepinephrine | 99.64 | 84.02 | 99.90 | 91.14 |
| D-Norepinephrine | 0.08 | 11.23 | n.d. | 8.26 |

n.d. = not detected

TABLE 6
Stability data at 25° C. for 0.16 mg/mL Norepinephrine in vials and IV bags that were not
nitrogen sparged (ambient air) and then autoclaved.

| | Storage at 25° C. | | | | | |
|---|---|---|---|---|---|---|
| | Vial | | | IV bags | | |
| Purity (%) | Initial | 3 Month | 6 Month | Initial | 3 Month | 6 Month |
| Norepinephrine | 96.65 | 90.68 | 83.95 | 98.65 | 94.20 | 89.53 |
| Total Impurities/RS | 3.35 | 9.31 | 16.05 | 1.36 | 5.78 | 10.48 |
| Chiral purity of | | | | | | |
| Norepinephrine (%) | Initial | 3 Months | 6 Month | Initial | 3 Months | 6 Month |
| L-Norepinephrine | 95.00 | 90.86 | 91.15 | 98.47 | 94.52 | n.a. |
| D-Norepinephrine | 2.85 | 5.02 | 4.34 | 0.64 | 2.66 | n.a. |

n.a. = not analyzed

TABLE 7
Stability data at 25° C. for 0.16 mg/mL Norepinephrine in vials and IV bags that were not
nitrogen sparged (ambient air) and not autoclaved.

| | Storage at 25° C. | | | | | |
|---|---|---|---|---|---|---|
| | Vial | | | IV bag | | |
| | Initial | 3 Month | 6 Month | Initial | 3 Month | 6 Month |
| Purity (%) | | | | | | |
| Norepinephrine | 99.70 | 96.25 | 92.54 | 99.78 | 95.41 | 91.60 |
| Total Impurities/RS | 0.31 | 3.74 | 7.45 | 0.22 | 4.58 | 8.19 |
| Chiral purity of | | | | | | |
| Norepinephrine (%) | | | | | | |
| L-Norepinephrine | 99.11 | 96.71 | 95.97 | 99.48 | 96.36 | 94.49 |
| D-Norepinephrine | 0.12 | 1.50 | 1.73 | — | 1.39 | 2.46 |

TABLE 8
Stability data for 0.04 mg/mL Norepinephrine in vials and IV bags that
were nitrogen sparged and autoclaved.

| | Storage at 25° C. | | | | | |
|---|---|---|---|---|---|---|
| | Vial | | | IV bag | | |
| | Initial | 3 Month | 6 Month | Initial | 3 Month | 6 Month |
| Purity (%) | | | | | | |
| Norepinephrine | 98.39 | 97.63 | 96.84 | 100.00 | 99.29 | 99.06 |
| Total Impurities/RS | 1.61 | 2.38 | 3.15 | 0.0 | 0.71 | 0.94 |
| Chiral purity of | | | | | | |
| Norepinephrine (%) | | | | | | |
| L-Norepinephrine | 97.16 | 94.26 | 94.24 | 99.20 | 98.39 | 97.54 |
| D-Norepinephrine | 2.08 | 3.51 | 3.48 | 0.53 | 0.92 | 1.39 |

| | Storage at 40° C. | | | |
|---|---|---|---|---|
| | Vial | | IV bag | |
| | Initial | 6 Month | Initial | 6 Month |
| Purity (%) | | | | |
| Norepinephrine | 98.39 | 70.51 | 100.00 | 90.44 |
| Total Impurities/RS | 1.62 | 29.5 | 0.0 | 9.56 |
| Chiral purity of | | | | |
| Norepinephrine (%) | | | | |
| L-Norepinephrine | 97.16 | 77.02 | 99.20 | 86.75 |
| D-Norepinephrine | 2.08 | 13.68 | 0.53 | 8.78 |

TABLE 9
Stability data for 0.04 mg/mL Norepinephrine in vials and IV bags that
were nitrogen sparged and not autoclaved.

| | Storage at 25° C. | | | | | |
|---|---|---|---|---|---|---|
| | Vials | | | IV bags | | |
| Purity (%) | Initial | 3 Month | 6 Month | Initial | 3 Month | 6 Month |
| Norepinephrine | 100.00 | NA | 97.35 | 99.22 | NA | 99.28 |
| Total Impurities/RS | 0.0 | NA | 2.64 | 0.78 | NA | 0.72 |
| Chiral purity of | | | | | | |
| Norepinephrine (%) | Initial | 3 Month | 6 Months | Initial | 3 Month | 6 Months |
| L-Norepinephrine | 99.44 | 97.05 | 96.77 | 99.78 | 98.99 | 98.27 |
| D-Norepinephrine | 0.16 | 1.14 | 1.31 | 0.11 | 0.57 | 0.84 |

| | Storage at 40° C. | | | |
|---|---|---|---|---|
| | Vials | | IV bags | |
| | Initial | 6 Month | Initial | 6 Month |
| Purity (%) | | | | |
| Norepinephrine | 100.00 | 73.88 | 99.22 | 93.41 |
| Total Impurities/RS | 0.0 | 26.12 | 0.78 | 6.58 |
| Chiral purity of | | | | |
| Norepinephrine (%) | | | | |
| L-Norepinephrine | 99.44 | 79.94 | 99.78 | 92.21 |
| D-Norepinephrine | 0.16 | 11.77 | 0.11 | 6.37 |

TABLE 10
Stability data for 0.04 mg/mL Norepinephrine in vials and IV bags that were not
nitrogen sparged (ambient air) and then autoclaved.

| | Storage at 25° C. | | | | | |
|---|---|---|---|---|---|---|
| | Vials | | | IV bags | | |
| | Initial | 3 Month | 6 Month | Initial | 3 Month | 6 Month |
| Purity (%) | | | | | | |
| Norepinephrine | 93.34 | NA | 37.07 | 96.58 | NA | 64.43 |
| Total Impurities/RS | 6.66 | NA | 62.93 | 3.42 | NA | 35.58 |
| Chiral purity of | | | | | | |
| Norepinephrine (%) | | | | | | |
| L-Norepinephrine | 89.01 | 78.29 | 70.58 | 91.80 | 90.00 | n.a. |
| D-Norepinephrine | 2.86 | 4.09 | 3.42 | 1.19 | 3.16 | n.a. |

n.a. = not analyzed

TABLE 11
Stability data for 0.04 mg/mL Norepinephrine in vials and IV bags that were not
nitrogen sparged (ambient air) and not autoclaved.

| | Storage at 25° C. | | | | | |
|---|---|---|---|---|---|---|
| | Vial | | | IV bag | | |
| | Initial | 3 Month | 6 Month | Initial | 3 Month | 6 Month |
| Purity (%) | | | | | | |
| Norepinephrine | 99.45 | n.a. | 81.19 | 96.39 | n.a. | 76.34 |
| Total Impurities/RS | 0.55 | n.a. | 18.82 | 3.61 | n.a. | 23.65 |
| Chiral purity of | | | | | | |
| Norepinephrine (%) | | | | | | |
| L-Norepinephrine | 97.95 | 93.40 | 92.28 | 96.46 | 85.31 | 81.30 |
| D-Norepinephrine | 0.21 | 2.40 | 2.21 | 0.17 | 5.13 | 7.92 |

n.a. = not analyzed

We claim:

1. A ready-to-administer stable injectable norepinephrine solution comprising water; norepinephrine, or a pharmaceutically acceptable salt thereof, in a concentration of from 0.04 to 0.16 mg/mL; and a tonicity agent, wherein the pH of the solution is from 3.7 to 4.3, wherein the solution is substantially free of chelating agents and antioxidants, and wherein the solution is contained within a first container.

2. The norepinephrine solution according to claim 1, wherein the norepinephrine or the pharmaceutically acceptable salt thereof is norepinephrine bitartrate.

3. The norepinephrine solution according to claim 2, wherein the norepinephrine bitartrate is present in a concentration of 0.16 mg/mL.

4. The norepinephrine solution according to claim 2, wherein the norepinephrine bitartrate is present in a concentration of 0.08 mg/mL.

5. The norepinephrine solution according to claim 2, wherein the norepinephrine bitartrate is present in a concentration of 0.04 mg/mL.

6. The norepinephrine solution according to claim 1, wherein the solution is free of pH adjusting agents.

7. The norepinephrine solution according to claim 1, wherein the tonicity agent is sodium chloride.

8. The norepinephrine solution according to claim 1, wherein the pH is from 4.0 to 4.3.

9. The norepinephrine solution according to claim 1, wherein the pH is from 3.7 to 4.0.

10. The norepinephrine solution according to claim 1, wherein the first container is contained within a second container, such that a space exists between the first container and the second container and the space is filled with an inert gas.

11. The norepinephrine solution according to claim 10, wherein the first container comprises a polypropylene bag and the second container comprises an aluminum pouch.

12. The norepinephrine solution according to claim 1, wherein the solution comprises at least 90% L-norepinephrine after storage for at least 3 months at room temperature as determined by chiral HPLC.

13. The norepinephrine solution according to claim 1, wherein the solution comprises less than 2% total impurities after storage for at least 3 months at room temperature as determined by HPLC.

14. A ready-to-administer stable injectable norepinephrine solution comprising:
water; norepinephrine bitartrate in a concentration of from 0.04 to 0.16 mg/ml; and 0.9% w/v sodium chloride, wherein the pH of the solution is from 3.7 to 4.3, wherein the solution is substantially free of chelating agents and antioxidants, wherein the solution is contained within an infusion bag, and wherein the infusion bag is contained within an aluminum pouch.

15. A process for producing a ready-to-administer stable injectable norepinephrine solution according to claim 1, comprising:
(a) combining a tonicity agent, water, and norepinephrine, or a pharmaceutically acceptable salt thereof, to obtain a composition with a concentration of norepinephrine of from 0.04 to 0.16 mg/mL;
(b) filling the composition into the first container; and
(c) sterilizing the composition to sterility to obtain the ready-to-administer stable injectable norepinephrine solution, wherein the pH of the solution is from 3.7 to 4.3 and wherein the solution is substantially free of chelating agents and antioxidants.

16. The process according to claim 15, further comprising placing the first container into a second container such that a space exists between the first container and the second container; and replacing the space between the first container and the second container with an inert gas.

17. The process according to claim 16, wherein the first container comprises a polypropylene bag and the second container comprises an aluminum pouch.

18. A method of controlling blood pressure in a patient in need thereof, comprising:
administering the ready-to-administer stable injectable norepinephrine solution according to claim 1 from the first container to the patient, wherein the solution is not diluted or reconstituted.

19. The method according to claim 18, wherein the tonicity agent is sodium chloride.

20. The method according to claim 18, wherein the norepinephrine or the pharmaceutically acceptable salt thereof is norepinephrine bitartrate.
